(19) United States
(12) Patent Application Publication
(10) Pub. No.: US 2011/0270227 A1
(43) Pub. Date: Nov. 3, 2011

(54) METHOD AND APPARATUS FOR THE ENTERAL DISPENSATION OF LIQUID MEDICATIONS

(75) Inventors: Megan Kleckner, Denver, CO (US); Steven Philip van Engen, Parker, CO (US); Shawna Reagan, Denver, CO (US); Kevin Wiggins, Denver, CO (US)

(73) Assignee: Baxa Corporation, Englewood, CO (US)

(21) Appl. No.: 12/905,775

(22) Filed: Oct. 15, 2010

Related U.S. Application Data
(60) Provisional application No. 61/330,131, filed on Apr. 30, 2010.

(51) Int. Cl. A61M 31/00; A61M 35/00

(52) U.S. Cl. 604/515; 604/500; 604/514; 604/290; 604/189; 604/311

(57) ABSTRACT

An enteral dispenser and associated method of use is provided for enterally dispensing liquid medications. The enteral dispenser includes a barrel having a number of dosing gradations arranged upon its outer surface. The barrel receives a liquid medication that has been selected from a predefined group of liquid medications having a range of colors. A plunger is moveably disposed within the barrel, and a piston is coupled to a distal end of the plunger. The piston has a peripheral portion for selective alignment with respect to a desired one of the dosing gradations and is a color that is optically differentiable from each of the colors represented within the range of colors of the predefined group of liquid medications, such that an user may differentiate between the color of the a peripheral portion of the piston and the color of any selected liquid medication.

### RELATED APPLICATIONS

This application claims priority to U.S. Provisional Application Ser. No. 61/330,131, filed Apr. 30, 2010, entitled “METHOD AND APPARATUS FOR THE ENTERAL DISPENSATION OF LIQUID MEDICATIONS”, the entirety of which is hereby incorporated by reference.

### FIELD OF THE INVENTION

The present invention relates generally to medical and pharmaceutical equipment, and more particularly, to the accurate dispensation of liquid medications using an enteral dispenser.

### BACKGROUND OF THE INVENTION

Each year, the healthcare industry uses countless enteral dispensers throughout the world for the oral, topical, rectal, vaginal, or sublingual administration of liquid medications to humans and animals. Such enterally administered liquid medications include, for example, liquid painkillers, cardiovascular drugs, steroids, barbiturates, enzymes, vitamins, minerals, and the like. Pharmaceutical manufacturers provide these liquid medications in variety of colors. For instance, enterally administered liquid painkillers are often variations of the colors pink, red, and orange, while liquid vitamins are often black. Other enterally administered liquid medications are blue, while still others may be clear.

Traditional enteral dispersers generally include a transparent barrel for receiving liquid medication through a barrel tip. The barrel generally includes a number of dosing gradations that are visible upon an outer surface of the barrel, while a plunger having a piston coupled with its distal end is moveably disposed within the barrel. To draw a dose of a selected liquid medication into the barrel, a user may insert the barrel tip into the selected liquid medication and withdraw the plunger until a predefined portion (e.g., a peripheral portion) of the piston is aligned with a desired dosing gradation.

Because enterally administered liquid medications come in a wide array of colors, it has been recognized that oftentimes either the peripheral portion of the piston and/or the dosing gradations are partially or completely obscured when the selected liquid medication blends in or matches the peripheral portion of the piston and/or the dosing gradations, making it more difficult for the user to visually align the peripheral portion of the piston with the appropriate dosing gradation. By way of example, the inventors have recognized that this issue is particularly prevalent in the dispensation of liquid medications that are the color black.

For one or a few enteral administrations of a liquid medication, the user might simply take special care to ensure accurate dosing. However, larger health care institutions often administer enteral medications to hundreds of patients per day, making it desirable for medical personnel be able to accurately fill enteral dispensers in an efficient and consistently repeatable manner, regardless of the color of the selected liquid medication.

### SUMMARY OF THE INVENTION

In view of the foregoing, a primary objective of the present invention is to provide apparatus and methods for enterally administering liquid medications.

Another objective of the present invention is to provide improved apparatus and methods for enterally administering liquid medications in a manner that results in accurate dosing.

Yet another objective of the present invention is to provide improved apparatus and methods for enterally administering liquid medications in a manner that is repeatable and time-efficient.

Yet another objective of the present invention is to provide an improved enteral dispenser that medical providers can generally stock for the dispensation of all or substantially all liquid medications.

One or more of the above-noted objectives and additional advantages may be realized by an inventive method for enterally dispensing liquid medication comprising selecting a liquid medication from a predefined group of liquid medications having a range of colors, where the range of colors may include the color black. The predefined group of liquid medications may include any appropriate combination of enterally administered liquid medications such as, for example, liquid painkillers, cardiovascular drugs, steroids, barbiturates, enzymes, vitamins, and minerals.

The method may further include drawing the selected liquid medication into a barrel of an enteral dispenser that is adapted to receive liquid medication. In this regard, a plunger may be moveably disposed within the barrel, and a piston may be coupled to a distal end of the plunger. Further, a number of dosing gradations may be arranged upon an outer surface of the barrel. The barrel, the piston, and the plunger of the enteral dispenser may be of any appropriate size and configuration to accommodate a desired dose of liquid medication and, as a result, the dosing gradations may vary appropriately according to the size of the enteral dispenser. Further, the barrel, piston, and plunger that form the enteral dispenser may comprise any appropriate material or combination of materials, including polymeric materials. By way of example, the barrel and the plunger may comprise a plastic material (e.g., polyethylene, polypropylene, etc.), and the piston may comprise, at least in part, an elastomeric or rubber material (e.g., polyethylene, silicone, etc.). In the latter regard, the piston may include at least one resilient peripheral portion (e.g., an outward facing peripheral surface) for slidably and sealably engaging an inside surface at the barrel. In one approach, the piston may comprise a resilient end cap member (e.g., a one-piece molded member) that may elastically deform during snap-fit engagement over a distal end of the plunger. In another approach the piston may comprise a resilient ring shaped member (e.g., an O-ring) that may elastically deform during positioning about an annular distal end of the plunger.

The step of drawing the selected liquid medication into the barrel of the enteral dispenser may comprise optically, or visually, differentiating between a color of the selected liquid medication and a color of the peripheral portion of the piston while aligning the peripheral portion of the piston relative to a desired one of the dosing gradations. The peripheral portion of the piston to be aligned with the desired dosing gradation may be preferably disposed about distal end portion. For instance, the peripheral portion may encircle a circumference of the piston at a distal end thereof. The dosing gradations may be arranged in relation to the peripheral portion of the piston for use in visual alignment.

In this regard, the inventors have recognized the desirability of enabling a user to optically differentiate between the color of the selected liquid medication and the color of the peripheral portion of the piston. This may allow the user to more accurately align the peripheral portion of the piston with the desired dosing gradation, thereby further increasing the accuracy with which liquid medication dosages are measured. This increase in accuracy improves the overall effectiveness of dispensing liquid medication enterally. Moreover, because the user may differentiate between the peripheral portion of the piston and the selected liquid medication, the user is able to quickly and confidently draw the selected medication into the enteral dispenser, improving the overall efficiency associated with using enteral dispensers for the administration of liquid medications. Still further, medical providers can stock one enteral dispenser for the dispensation of all of the liquid medications within the predefined group of liquid medications, simplifying the procurement, stocking, and tracking demands placed on the medical provider.

In addition to the color black, one implementation of the range of colors represented within the predefined group of liquid medications may include clear as well as shades of the colors pink, red, orange, and dark blue. In this implementation, the color of the peripheral portion of the piston may be a shade of the color gray. Alternatively, it may be a shade of the colors violet, green, yellow, white, or light blue. Notably, while a shade of gray is presently believed preferable, the piston may be any color or color shade that is optically differentiable from each of the colors represented within the range of colors of the predefined group of liquid medications, and the predefined group of liquid medications may be defined in any manner that is customary, appropriate, and/or necessary.

The step of drawing the selected liquid medication into the barrel of the enteral dispenser, discussed above, may further include optically, or visually, differentiating between the color of the selected liquid medication and a color of the dosing gradations. Similar to optically differentiating between the selected liquid medication and the color of the peripheral portion of the piston, optically differentiating between the selected liquid medication and the color of the dosing gradations further improves the user's ability to align the peripheral portion of the piston with the desired dosing gradation, resulting in additional advances in accurately dosing liquid medications for enteral administration.

In one embodiment in which the range of colors represented within the predefined group of liquid medications includes black, clear, and shades of pink, red, orange, and dark blue, the dosing gradations may be a shade of light blue or, similar to the peripheral portion of the piston, they may be any other color that is optically differentiable from each of the colors represented within the range of colors of the predefined group of liquid medications. Moreover, the dosing gradations may be the same or a different color than the color of the peripheral portion of the piston.

According to another aspect, a dispenser for enterally administering liquid medications may be provided. The dispenser may include a barrel having a number of dosing gradations arranged upon an outer surface of the barrel. The barrel may be adapted to receive a liquid medication that is selected from a predefined group of liquid medications having a range of colors that may include the color black. The predefined group of liquid medications may include any appropriate combination of enterally administered liquid medications, including, for example, liquid painkillers, cardiovascular drugs, steroids, barbiturates, enzymes, vitamins, and minerals.

The dispenser may also include a plunger having a proximate end and a distal end. The plunger may be moveably disposed within the barrel. In addition, a piston having a peripheral portion for selective alignment with respect to one of the dosing gradations may be coupled to the distal end of the plunger. The barrel may further include a barrel tip that is adapted for one of a plurality of enteral dispensing applications including, for example, oral dispensation, topical dispensation, rectal dispensation, vaginal dispensation, and sublingual dispensation. In this regard, the barrel tip may be specifically configured to prevent connection with a luer needle hub or a needle-less IV connector, thereby decreasing the possibility that the enteral dispenser will be used for non-enteral applications. In one embodiment, the barrel tip may be free of external or internal threading, and in another implementation, the barrel tip may have a diameter that is larger than the diameter of conventional syringe luer-tips so as to prevent the barrel tip of the enteral dispenser from successfully mating with a luer needle hub or needle-less IV connector. Further, in one implementation, a tip cap may be placed upon the barrel tip when the dispenser is not in use.

As discussed above, the barrel and the barrel tip, the tip cap, the piston, and the plunger of the enteral dispenser may be of any appropriate size and/or configuration to accommodate a desired dose of liquid medication and, as a result, the dosing gradations may vary according to the size of the enteral dispenser. Moreover, the barrel, the barrel tip, the piston, and the plunger that form the enteral dispenser may comprise any appropriate material or combination of materials.

In further relation to this aspect, at least a peripheral portion of the piston may have a color that is optically differentiable from each color represented within the range of colors of the predefined group of liquid medications. For instance, in addition to the color black, one variation of the range of colors of the predefined group of liquid medications may include clear as well as shades of the colors pink, red, orange, and dark blue. In this variation, at least the peripheral portion of the piston may be a shade of gray. Alternatively, the peripheral portion of the piston may be any other color that is optically differentiable from each of the colors represented within the predefined group of liquid medications, such as, for example, a shade of violet, green, yellow, white, or light blue. Notably, the predefined group of liquid medications may include any appropriate enterally administered liquid medications of any color or color shade.

By virtue of the noted color features of the liquid medications within the predefined group of liquid medications and the peripheral portion of the piston, the inventive enteral dispenser promotes accurate dosing of liquid medications. That is, because an user is able to more easily see the peripheral portion of the piston through any selected liquid medication that has been drawn into the barrel, the user can more accurately align the peripheral portion of the piston with respect to the desired dosing gradation. The user can also perform the task of filling the enteral dispenser in less time, rendering the enteral administration process more efficient.

In one embodiment, the dosing gradations arranged upon the outer surface of the barrel of the enteral dispenser may also have a color that is optically differentiable from each color that is represented within the range of colors of the predefined group of liquid medications. For instance, in a variation in which the range of colors of the predefined group of liquid medications includes black, clear, and shades of the colors pink, red, orange, and dark blue, the dosing gradations may be light blue. Alternatively, they may be any other color that is optically differentiable from each of the colors represented within the predefined group of liquid medications, such as, for example, a shade of violet, green, yellow, white, or gray. The dosing gradations may be the same or a different color than the color of the piston.

Additional features and advantages of the present invention will become apparent to those skilled in the art upon review of the further description hereinbelow.

Numerous additional features and advantages of the present invention will become apparent to those skilled in the art upon consideration of the embodiment descriptions provided hereinbelow.

### BRIEF DESCRIPTION OF THE DRAWINGS

FIG. 1 shows a front exploded view of one embodiment of an enteral dispenser.

FIG. 2A shows a front view of another embodiment of an enteral dispenser as a plunger is withdrawn to draw a liquid medication into a barrel of the enteral dispenser.

FIG. 2B shows a front view of the enteral dispenser embodiment of FIG. 2A as the plunger is compressed to dispense the liquid medication from the barrel of the enteral dispenser.

FIG. 3 shows a schematic that graphically demonstrates one embodiment of a group of predefined liquid medications and the resulting color options for embodiments of at least a peripheral portion of a piston and/or dosing gradations of an enteral dispenser.

FIG. 4 is a flow diagram showing a method for enterally dispensing a liquid medication from the enteral dispenser embodiments of FIGS. 1 and 2A-B.

### DETAILED DESCRIPTION

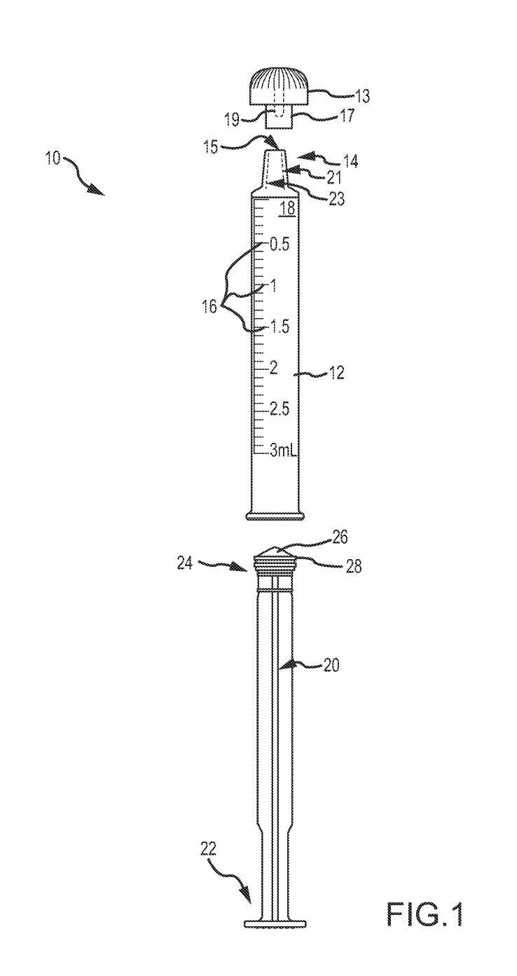

FIG. 1 illustrates an exemplary enteral dispenser 10 for the enteral administration or dispensation of liquid medications such as liquid painkillers, cardiovascular drugs, steroids, barbiturates, enzymes, vitamins, minerals, and the like. Such enteral administration includes the oral, topical, rectal, vaginal, and/or sublingual administration of liquid medications.

The enteral dispenser 10 includes a barrel 12 and a hollow barrel tip 14. The hollow barrel tip 14 includes an opening 15 for passage of a liquid medication (not shown) therethrough. When the enteral dispenser 10 is not in use, a tip cap 13 may be placed upon the barrel tip 14. The tip cap 13 includes an outer sleeve 17 and an internal shaft 19. To engage the tip cap 13 with the barrel tip 14, an user may press the tip cap 13 onto the barrel tip 14 such that the outer sleeve 17 is disposed about an outer surface 21 of the barrel tip 14, while the internal shaft 19 extends through the opening 15 of the barrel tip 14 and frictionally engages a tapered inner surface 23 of the barrel tip 14. In this regard, the tip cap 13 frictionally seals the opening 15 of the barrel tip 14 to prevent leakage and prolong medication life.

The barrel tip 14 of the enteral dispenser 10 is specifically designed for use in enterally administrating (e.g., orally administering) liquid medications. In this regard, the barrel tip 14 is configured to prevent tubing misconnections and cannot connect to a luer needle hub or activate a needle-less IV connector or port. More specifically, both the outer surface 21 and the inner surface 23 of the barrel tip 14 are free of threading or other projections and cannot threadably engage with a luer needle or needle-less IV connector. In addition, the barrel tip 14 has an outer diameter that is larger than a diameter of a conventional luer-tip syringe. As a result, the barrel tip 14 cannot successfully mate with a luer needle hub or needle-less IV port. Thus, the enteral dispenser 10 provides for safe and accurate delivery of enterally administered liquid medications and reduces the possibility of wrong-route administration through, for example, a patient's IV or epidural line.

A number of dosing gradations 16 are arranged along an outer surface 18 of the barrel 12. The dosing gradations 16 may be applied to the barrel 12 in any appropriate manner. For instance, in one embodiment, the dosing gradations 16 may be printed upon the outer surface 18 of the barrel 12. In addition, the barrel 12 may be of any appropriate size so as to accommodate a range of desired doses of liquid medication. As a result, the number of dosing gradations 16 and the volume values reflected by the dosing gradations 16 may change as a function of the size of the barrel 12. As will be further described, the gradations 16 may be of a color selected for differentiation in relation to liquid medications to be administered using the dispenser 10.

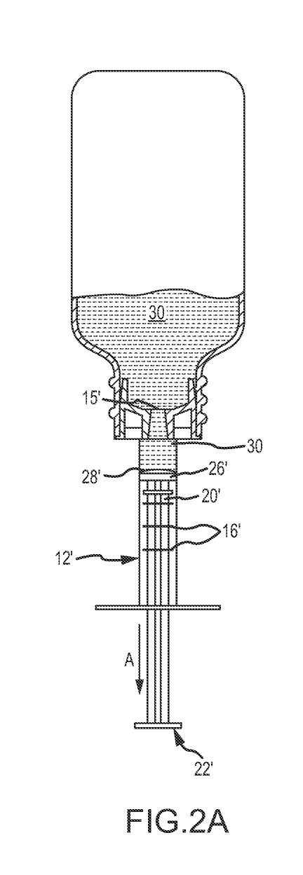

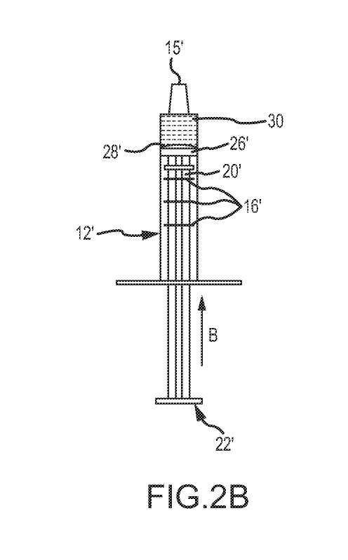

The enteral dispenser 10 also includes a plunger 20 having proximate and distal ends 22, 24. The distal end 22 of the plunger 20 terminates in a piston 26 such that when the plunger 20 is moveably disposed within the barrel 12, piston 26 acts as a seal between the liquid medication (not shown) contained with the barrel 12 and the plunger 20. The piston 26 also acts to transfer force applied to the proximate end 22 of the plunger 20 to the liquid medication, thereby expelling or dispensing the liquid medication from the barrel 12, as discussed below with reference to FIGS. 2A-B.

The piston 26 includes a peripheral portion, or rim, 28 for alignment with a desired one of the dosing gradations 16. That is, when the peripheral portion 28 of the piston 26 is aligned with one of the dosing gradations 16, a volume of liquid medication that corresponds to the volume reflected by the dosing gradation 16 has been drawn into the barrel 12 of the dispenser 10. As will be further described, the peripheral portion 28 may be of a color selected for differentiation in relation to liquid medications to be administered using the dispenser 10. In one implementation, the peripheral portion 28 is a shade of the color gray.

The barrel 12, the plunger 20, the piston 26, and the tip cap 13 may be formed of any appropriate material or combination of materials. In one embodiment, the barrel 12, the plunger 20, and the tip cap 13 may each be formed of a disposable plastic such as, for instance, polyethylene or polypropylene. The piston may be formed of any appropriate resilient, or elastomeric, material including e.g., polyethylene, silicone, or rubber.

In the illustration embodiment, the piston 26 and peripheral portion 28 thereof are integrally defined by a one-piece, molded cap member. The cap member is conically configured at its distal end and provided with two outer, peripheral flanges along a body length thereof. As illustrated, the most distal flange defines the peripheral portion 28.

In an alternate embodiment, a peripheral portion of a piston may be defined by a ring-shaped, or donut-shaped, member located about a distal end of a plunger. For example, an elastomeric O-ring may be employed.

FIGS. 2A-B show a variation of an enteral dispenser 10′ as liquid medication is drawn into the dispenser 10′ (FIG. 2A) and as liquid medication is expelled from the dispenser 10′ (FIG. 2B). Component illustrations of the enteral dispenser embodiment 10′ that correspond to the enteral dispenser 10 are identified by the same reference numerals used in connection the enteral dispenser 10, followed by a “single prime” designation.

To draw a liquid medication 30 into a barrel 12′ of the enteral dispenser 10′, as shown in FIG. 2A, an user may place an opening 15′ of the barrel tip 14′ into contact with the liquid medication 30 contained within a receptacle (e.g., a bottle or vial) and withdraw a plunger 20′ from an initial position in which a piston 26′ is at or near the barrel tip 14′ in the direction of arrow A. The user may continue to withdraw the plunger 20′ until a peripheral portion 28′ of the piston 26′ aligns with one of several dosing gradations 16′. Conversely, the user may apply a force to the proximate end 22′ of the plunger 20′ to move the plunger 20′ in the direction of arrow B, as shown in FIG. 2B. This movement decreases the volume between the opening 15′ of the barrel tip 14′ and the piston 26′, thereby expelling or dispensing the liquid medication 30 from the barrel tip 14′.

As discussed above, liquid medications are available in a range of colors. For example, liquid medications that are designed to treat a particular ailment are often produced in a specific color or in a range of shades that fall within a common base color (e.g., various shades of red, various shades of orange). For example, liquid painkillers are often produced in shades of the colors pink, red, and orange, and liquid vitamin supplements are often produced in the color black. In addition, liquid medication colors may vary according to the pharmaceutical manufacturer.

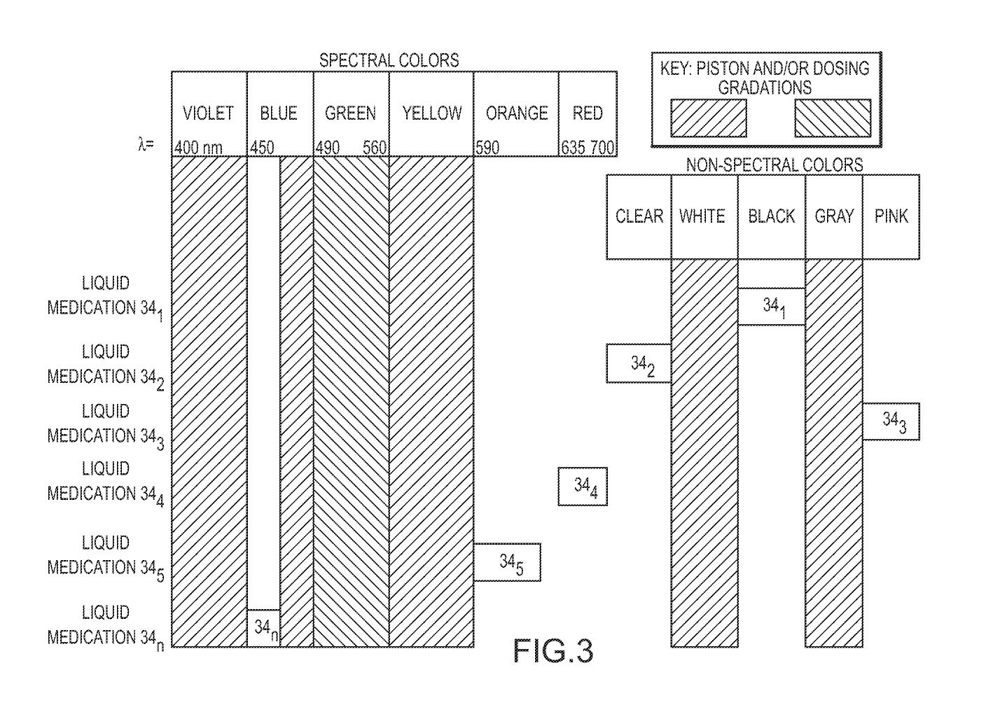

To carry out an administration of liquid medication as shown in FIGS. 2A-B, the user (FIGS. 2A-B) may first select a liquid medication from among a predefined group of liquid medications 34₁₋ₙ, which is graphically depicted in FIG. 3. Within the predefined group of liquid medications 34₁₋ₙ, each of the liquid medications 34₁₋ₙ may be a different color or color shade. A first liquid medication 34₁ may be black; a second liquid medication 34₂ may be clear; a third liquid medication 34₃ may be a shade of pink; a fourth liquid medication 34₄ may be a shade of red; a fifth liquid medication 34₅ may be a shade of orange; and a sixth liquid medication 34ₙ may be a shade of blue. For more particular demonstrative purposes, the sixth liquid medication 34ₙ may be a shade within the particular wavelength interval for dark blue.

As noted in FIG. 3, some of the liquid medication color shades are within the range of spectral colors, which are evoked by a single wavelength of light in the visible spectrum (e.g., violet, blue, green, yellow, orange, and red), while others are non-spectral colors such as achromatic colors (e.g., black) and/or colors created by mixing other spectral and/or non-spectral colors (e.g., pink as a mixture of the spectral color red and the non-spectral color white). Still other liquid medications lack any pigment whatsoever (e.g., the second liquid medication 34₂ is clear). To ensure cohesiveness, these various options are equally referred to as “colors” throughout this discussion.

In aligning the distal edge 28, 28′ of the piston 26, 26′ with the appropriate dosing gradation 16, 16′, as discussed above, the user optically differentiates between a color of the liquid medication 34₁₋ₙ selected for administration and a color of the peripheral portion 28, 28′ of the piston 26, 26′. If the color of the selected liquid medication 34₁₋ₙ matches or blends in with the color of the peripheral portion 28, 28′ of piston 26, 26′, such alignment is more difficult to perform because the selected liquid medication 34₁₋ₙ obscures the peripheral portion 28, 28′ of piston 26, 26′. For instance, if the peripheral portion 28, 28′ of piston 26, 26′ is black, and the user selects the liquid medication 34₁ (i.e., a black liquid medication) for administration using the enteral dispenser 10, 10′, the liquid medication 34₁ may obscure the peripheral portion 28, 28′ of piston 26, 26′ and the user may experience difficulty differentiating between the peripheral portion 28, 28′ of piston 26, 26′ and the selected liquid medication 34₁ when drawing the liquid medication 34₁ into the barrel 12, 12′. As a result, the user may not be able to accurately align the peripheral portion 28, 28′ of the piston 26, 26′ with the appropriate dosing gradation 16, 16′ because the user cannot clearly observe the peripheral portion 28, 28′ of the piston 26, 26′. At the very least, the user may require additional time to visually align the peripheral portion 28, 28′ of the piston 26, 26′ with an appropriate one of the dosing gradations 16, 16′.

To address this disadvantage and promote efficient and accurate manual alignment of the peripheral portion 28, 28′ of the piston 26, 26′ and the desired dosing gradation 16, 16′, one embodiment of the enteral dispenser 10, 10′ includes a peripheral portion 28, 28′ of piston 26, 26′ having a color that is optically differentiable from (i.e., that optically contrasts) each color represented within the range of colors of the predefined group of liquid medications 34₁₋ₙ. For example, FIG. 3 illustrates that multiple piston colors would render the peripheral portion 28, 28′ of the piston 26, 26′ optically differentiable from each of the colors represented within the exemplary predefined group of liquid medications 34₁₋ₙ. That is, as shown in FIG. 3, the peripheral portion 28, 28′ of the piston 26, 26′ would contrast each of the liquid medications 34₁₋ₙ if the peripheral portion 28, 28′ of the piston 26, 26′ were a shade of violet (reflecting light having a wavelength between approximately 400-450 nm), green (reflecting light having a wavelength between approximately 490-560 nm), yellow (reflecting light having a wavelength between approximately 560-590 nm), white, or gray. In addition, shades of light blue (reflecting light having a wavelength between approximately 475-490) would also contrast the colors of each of the liquid medications 34₁₋ₙ in a manner similar to the optical effect of, for example, white or gray. Thus, the user could choose any of the liquid medications 34₁₋ₙ within the group of predefined liquid medications 34₁₋ₙ and, regardless of the user's selection, the peripheral portion 28, 28′ of the piston 26, 26′ would be equally optically differentiable from the selected liquid medication 34₁₋ₙ.

Beyond the peripheral portion 28, 28′ of the piston 26, 26′, the dosing gradations 16, 16′ may also have a color that is optically differentiable from the color of each of the liquid medications 34₁₋ₙ of the group of predefined liquid medications 34₁₋ₙ. The color of the dosing gradations 16, 16′ may match the color of the peripheral portion 28, 28′ of the piston 26, 26′ or it may be another differentiable color. For instance, in one embodiment, both the peripheral portion 28, 28′ of the piston 26, 26′ and the dosing gradations 16, 16′ may be a shade of light blue. In another embodiment, the peripheral portion 28, 28′ of the piston 26, 26′ may be a shade of gray, while the dosing gradations 16, 16′ may be a shade of light blue. Any appropriate piston/dosing gradation color-combination may be used to promote ease in optical differentiation between the selected liquid medication 34₁₋ₙ and the peripheral portion 28, 28′ of the piston 26, 26′ and/or the selected liquid medication 34₁₋ₙ and the dosing gradations 16, 16′.

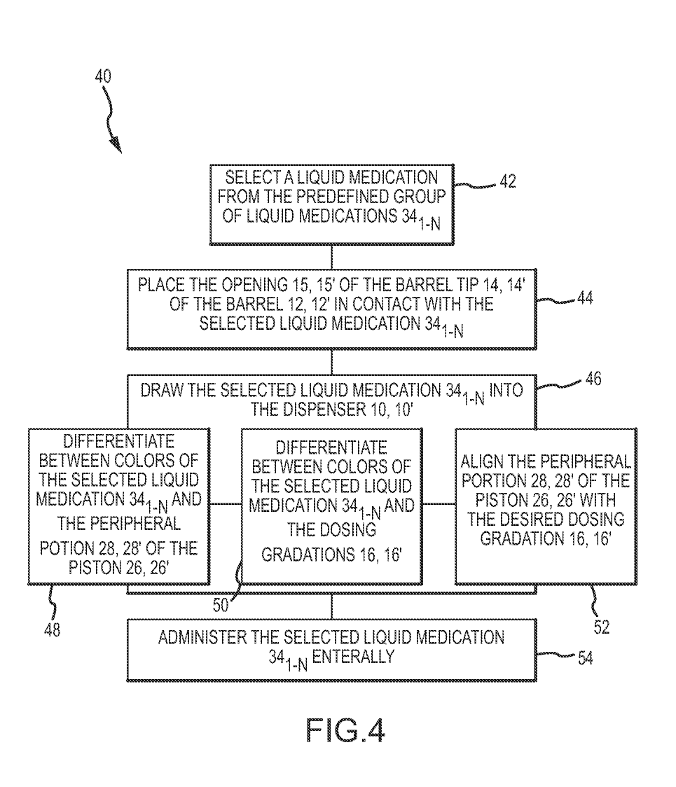

FIG. 4 presents a flow diagram showing an exemplary method 40 for enterally dispensing or administering a liquid medication selected from the predefined group of liquid medications 34₁₋ₙ from the enteral dispenser 10, 10′. First, the user selects a liquid medication from among the predefined group of liquid medications 34₁₋ₙ (step 42). Next, the user places the opening 15, 15′ of the barrel tip 14, 14′ of the enteral dispenser 10, 10′ into contact with the selected liquid medication 34₁₋ₙ (step 44) and draws the selected liquid medication 34₁₋ₙ into the enteral dispenser 10, 10′ (step 46). Drawing the selected liquid medication 34₁₋ₙ into the enteral dispenser 10, 10′ (step 46) includes three concurrent steps: The user optically differentiates between the color of the selected liquid medication 34₁₋ₙ and the color of the piston 16, 16′ (step 48) and optically differentiates between the color of the selected liquid medication 34₁₋ₙ and the color of the dosing gradations 16, 16′ (step 50) while withdrawing the plunger 20, 20′ until the peripheral portion 28, 28′ of the piston 26, 26′ aligns with the desired one of the dosing gradations 16, 16′ (step 52). After drawing the selected liquid medication 34₁₋ₙ into the enteral dispenser 10, 10′, the user compresses the plunger 20, 20′ to enterally dispense the selected liquid medication 34₁₋ₙ to a patient according to one of a number of enteral applications including oral dispensation, topical dispensation, rectal dispensation, vaginal dispensation, and sublingual dispensation (step 54).

The enteral dispenser 10, 10′ may be packaged in a remote production location (not shown), either alone or as a kit including multiple dispensers 10, 10′, and each of the dispensers 10, 10′ may be sterilized, either before or after being packaged. In one implementation, the selected liquid medication 34₁₋ₙ may be chosen at the production location, and the dispensers 10, 10′ may be packaged along with a vial or other receptacle containing the selected liquid medication 34₁₋ₙ. In this embodiment, upon receiving the package containing the one or more dispensers 10, 10′ and the selected liquid medication 34₁₋ₙ, the user may remove the packaging and uncap the dispenser 10, 10′ before filling the dispenser with the selected liquid medication 34₁₋ₙ. The user may then replace the cap and transfer the filled dispenser 10, 10′ to a patient location (not shown) before uncapping and dispensing the selected liquid medication 34₁₋ₙ, as discussed above. Finally, once the selected liquid medication 34₁₋ₙ has been dispensed, the dispenser 10, 10′ may be disposed of in any appropriate manner, either at the patient location or another, separate location.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. A method for enterally dispensing liquid medication, said method comprising:
selecting any liquid medication from a predefined group of liquid medications having a range of colors, said range of colors including the color black; and
drawing said selected liquid medication into a barrel of an enteral dispenser that is adapted to receive liquid medication, wherein a plunger is moveably disposed within said barrel, a piston is coupled to a distal end of said plunger, and a number of dosing gradations are arranged upon an outer surface of said barrel, said step of drawing comprising:
optically differentiating between a color of said selected liquid medication and a color of a peripheral portion of said piston, wherein said color of the peripheral portion is established for differentiation in relation to said range of colors of said predetermined group of medications; and
during said step of differentiating, aligning an edge of said piston relative to a desired one of said dosing gradations.

2. The method of claim 1, further comprising:
enterally administering said selected liquid medication, wherein said enterally administering comprises one of orally dispensing, topically dispensing, rectally dispensing, vaginally dispensing, and sublingually dispensing.

3. The method of claim 1, wherein said predefined group of liquid medications includes liquid painkillers, cardiovascular drugs, steroids, barbiturates, enzymes, vitamins, and minerals.

4. The method of claim 1, wherein said color of said a peripheral portion of said piston is a shade of the color gray.

5. The method of claim 4, wherein said range of colors of said predefined group of liquid medications further includes clear and shades of the colors pink, red, orange, and dark blue.

6. The method of claim 5, wherein said step of drawing further comprises:
optically differentiating between said color of said selected liquid medication and a color of said dosing gradations, wherein said color of said dosing gradations is established for differentiation in relation to said range of colors of said predetermined group of medications.

7. The method of claim 6, wherein said color of said peripheral portion and said color of said dosing gradations are the same.

8. The method of claim 6, wherein said color of said peripheral portion and said color of said dosing gradations are different.

9. The method of claim 1, wherein said peripheral portion of said piston comprises said edge of the piston.

10. The method of claim 9, wherein said piston is integrally defined by a one-piece, molded member.

11. The method of claim 9, wherein said peripheral portion is defined by a ring-shaped member.

12. The method of claim 1, further comprising:
packaging said enteral dispenser and a receptacle containing the selected liquid medication in a package.

13. The method of claim 12, wherein said packaging step is completed at a first location, and further comprising:
removing the enteral dispenser and receptacle from the package at a second location, remote from said first location;
completing said drawing step after said removing step; and,
enterally administering said selected liquid medication after said completing step, wherein said enterally administering comprises one of orally dispensing, topically dispensing, rectally dispensing, vaginally dispensing, and sublingually dispensing.

14. The method of claim 1, wherein said color of said a peripheral portion of said piston is a shade of the color gray.

15. A dispenser for enterally administering liquid medications, comprising:
a barrel having a number of dosing gradations arranged upon an outer surface of said barrel, wherein said barrel is adapted to receive any liquid medication selected from a predefined group of liquid medications having a range of colors, and wherein said range of colors includes the color black;
a plunger movably disposed within said barrel, said plunger having a proximate end and a distal end; and
a piston coupled to said distal end of said plunger, said piston having a peripheral portion for selective alignment with respect to any one of said dosing gradations and a color established to be optically differentiable from each color represented within said range of colors of said predefined group of liquid medications.

16. The dispenser of claim 15, wherein said barrel further comprises a barrel tip, wherein said barrel tip is adapted for one of a plurality of enteral dispensing applications including oral dispensation, topical dispensation, rectal dispensation, vaginal dispensation, and sublingual dispensation.

17. The dispenser of claim 15, wherein said predefined group of liquid medications includes liquid painkillers, cardiovascular drugs, steroids, barbiturates, enzymes, vitamins, and minerals.

18. The dispenser of claim 15, wherein said dosing gradations have a color established to be optically differentiable from each color represented within said range of colors of said predefined group of liquid medications.

19. The dispenser of claim 18, wherein said color of said peripheral portion and said color of said dosing gradations are the same.

20. The dispenser of claim 18, wherein said color of said peripheral portion and said color of said dosing gradations are different.

21. The dispenser of claim 18, wherein said color of said peripheral portion of said piston is a shade of the color gray.

22. The dispenser of claim 21, wherein said range of colors of said predefined group of liquid medications further includes clear and shades of the colors pink, red, orange, and dark blue.

23. The dispenser of claim 15, wherein said color of said a peripheral portion of said piston is a shade of the color gray.

24. The dispenser of claim 15, wherein said piston is integrally defined by a one-piece, molded member.

25. The dispenser of claim 15, wherein said peripheral portion is defined by a ring-shaped member.

26. The dispenser of claim 15, wherein said peripheral portion comprises an elastomeric material.
